(19) United States
(12) Patent Application Publication
(10) Pub. No.: US 2025/0352230 A1
(43) Pub. Date: Nov. 20, 2025

(54) THROMBUS ASPIRATION CONTROL SYSTEMS AND RELATED METHODS FOR CONTROLLING BLOOD LOSS

(71) Applicant: CONTEGO MEDICAL, INC., Raleigh, NC (US)

(72) Inventor: Christopher R. Kilgus, Felton, CA (US)

(21) Appl. No.: 19/175,722

(22) Filed: Apr. 10, 2025

Related U.S. Application Data
(60) Provisional application No. 63/649,619, filed on May 20, 2024.

(51) Int. Cl. A61B 17/221; A61B 17/22; A61M 1/00; A61M 39/28

(52) U.S. Cl. CPC A61B 17/221; A61M 1/743; A61M 1/75; A61M 39/281; A61B 2017/22079; A61M 2205/3334; A61M 2205/3375

(57) ABSTRACT

Aspiration control systems and methods for controlling blood loss during thrombus removal are disclosed herein. An example system includes a receptacle; an aspiration tubing configured to fluidically couple the receptacle to an aspiration catheter; a vacuum line configured to fluidically couple the receptacle to a vacuum source; a sensor configured to measure a flow parameter associated with a liquid within the aspiration tubing; a vacuum regulator fluidically coupled to the vacuum line; and a controller operably coupled to the sensor and the vacuum regulator. The controller is configured to receive the flow parameter from the sensor, determine a flow condition based on the flow parameter, set a characteristic of a pulsed control signal based on the flow condition, and transmit the pulsed control signal to the vacuum regulator, the pulsed control signal being configured to control the vacuum regulator to adjust a vacuum level within the vacuum line.

### CROSS REFERENCE TO RELATED APPLICATIONS

This application claims the benefit of U.S. Provisional Application No. 63/649,619, filed May 20, 2024, which is incorporated herein by reference in its entirety for all purposes.

### FIELD

This invention relates to medical devices for thrombus removal, and more particularly to thrombus aspiration control systems.

### BACKGROUND

It is often desirable to remove tissue from the body in a minimally invasive manner as possible, so as not to damage other tissues. For example, removal of tissue (e.g., blood clots) from the vasculature may improve patient conditions and quality of life.

Many vascular system problems stem from insufficient blood flow through blood vessels. One of the causes of insufficient or irregular blood flow is a blockage within a blood vessel referred to as a blood clot, or thrombus. Thrombi can occur for many reasons, including after a trauma such as surgery, or due to other causes. For example, a large percentage of the more than 1.2 million heart attacks in the United States are caused by blood clots (thrombi) which form within a coronary artery.

When a thrombus forms, it may effectively stop the flow of blood through the zone of formation. If the thrombus extends across the interior diameter of an artery, it may cut off the flow of blood through the artery. If one of the coronary arteries is 100% thrombosed, the flow of blood is stopped in that artery, resulting in a shortage of oxygen carrying red blood cells, e.g., to supply the muscle (myocardium) of the heart wall. Such a thrombosis is unnecessary to prevent loss of blood but can be undesirably triggered within an artery by damage to the arterial wall from atherosclerotic disease. Thus, the underlying disease of atherosclerosis may not cause acute oxygen deficiency (ischemia) but can trigger acute ischemia via induced thrombosis. Similarly, thrombosis of one of the carotid arteries can lead to stroke because of insufficient oxygen supply to vital nerve centers in the cranium. Oxygen deficiency reduces or prohibits muscular activity, can cause chest pain (angina pectoris), and can lead to death of myocardium which permanently disables the heart to some extent. If the myocardial cell death is extensive, the heart will be unable to pump sufficient blood to supply the body's life sustaining needs. The extent of ischemia is affected by many factors, including the existence of collateral blood vessels and flow which can provide the necessary oxygen.

Clinical data indicates that clot removal may be beneficial or even necessary to improve outcomes. For example, in the peripheral vasculature, inventions and procedures can reduce the need for an amputation by 80 percent. The ultimate goal of any modality to treat these conditions of the arterial or venous system is to remove the blockage or restore patency, quickly, safely, and cost effectively. This may be achieved by thrombus dissolution, fragmentation, thrombus aspiration or a combination of these methods.

Catheter directed thrombectomy and thrombolysis are commonly perceived to be less traumatic, less likely to decrease the morbidity and mortality associated with conventional surgical techniques. In recent years, direct administration of chemical lysing agents into the coronary arteries has shown to be of some benefit to patients who have thrombosed coronary arteries. In this procedure, a catheter is placed immediately in front of the blockage and a drip of streptokinase is positioned to be directed at the upstream side of the thrombus. Streptokinase is an enzyme which is able in time to dissolve the fibrin molecule. This procedure can take several hours and is not always successful in breaking up the thrombus. Furthermore, it can lead to downstream thrombus fragments (emboli) which can lead to blockage of small diameter branches.

Thrombectomy is a technique for mechanical removal of blood clots in an artery or vein. It refers to physically removing a clot as opposed to employing chemical lysis to dissolve it. Multiple devices have been introduced to break up and remove clot and plaque, but each has its own shortcomings. Specifically, the existing systems do not provide adequate methods for breaking up the clot into smaller pieces for subsequent aspiration. Also, they do not provide a method for removing the thrombectomy device over a guidewire and reinserting into the same location to complete the procedure. Furthermore, conventional aspiration systems offer little control over the amount of blood lost during the procedure. There is a need for improved thrombectomy devices and aspiration systems that are safer and more effective for removing thrombus and plaque from the vascular system.

### SUMMARY

In some aspects, the disclosure relates to an aspiration control system including: a receptacle; an aspiration tubing configured to fluidically couple the receptacle to an aspiration catheter; a vacuum line configured to fluidically couple the receptacle to a vacuum source; a sensor configured to measure a flow parameter associated with a liquid within the aspiration tubing; a vacuum regulator fluidically coupled to the vacuum line; and a controller operably coupled to the sensor and the vacuum regulator, wherein the controller is configured to: receive the flow parameter from the sensor, determine a flow condition based on the flow parameter, set a characteristic of a pulsed control signal based on the flow condition, and transmit the pulsed control signal to the vacuum regulator, the pulsed control signal being configured to control the vacuum regulator to adjust a vacuum level within the vacuum line.

In some aspects, the disclosure relates to an aspiration control system, wherein: in response to determining a high flow condition, the characteristic of the pulsed control signal is set to a first value, and in response to determining a low flow condition, the characteristic of the pulsed control signal is set to a second value, wherein the second value is different than the first value.

In some aspects, the disclosure relates to an aspiration control system, wherein the characteristic of the pulsed control signal is a pulse duration.

In some aspects, the disclosure relates to an aspiration control system, wherein: in response to determining a high flow condition, the pulse duration is set to a first value, and in response to determining a low flow condition, the pulse duration is set to a second value, wherein the second value is greater than the first value.

In some aspects, the disclosure relates to an aspiration control system, wherein the first value is about 0.45 seconds, and the second value is about 0.80 seconds.

In some aspects, the disclosure relates to an aspiration control system, wherein the characteristic of the pulsed control signal is a pulse frequency.

In some aspects, the disclosure relates to an aspiration control system, wherein: in response to determining a high flow condition, the pulse frequency is set to a first value, and in response to determining a low flow condition, the pulse frequency is set to a second value, wherein the first value is greater than the second value.

In some aspects, the disclosure relates to an aspiration control system, wherein the first value is about 22 bursts per minute, and the second value is about 19 bursts per minute.

In some aspects, the disclosure relates to an aspiration control system, wherein the characteristic of the pulsed control signal is a duty cycle.

In some aspects, the disclosure relates to an aspiration control system, wherein: in response to determining a high flow condition, the duty cycle is set to a first value, and in response to determining a low flow condition, the duty cycle is set to a second value, wherein the second value is greater than the first value.

In some aspects, the disclosure relates to an aspiration control system, wherein the first value is about 20%, and the second value is about 25%.

In some aspects, the disclosure relates to an aspiration control system, wherein the flow condition is determined by comparing the flow parameter to a threshold value.

In some aspects, the disclosure relates to an aspiration control system, wherein the threshold value is between about 70 mL/min and about 130 mL/min.

In some aspects, the disclosure relates to an aspiration control system, wherein the threshold value is about 100 mL/min.

In some aspects, the disclosure relates to an aspiration control system, wherein the flow parameter is greater than or equal to the threshold value in a high flow condition.

In some aspects, the disclosure relates to an aspiration control system, wherein the flow parameter is less than the threshold value in a low flow condition.

In some aspects, the disclosure relates to an aspiration control system, wherein the vacuum regulator is arranged along the vacuum line between the vacuum source and the receptacle.

In some aspects, the disclosure relates to an aspiration control system, wherein the vacuum regulator includes a control valve.

In some aspects, the disclosure relates to an aspiration control system, wherein the vacuum regulator is configured to manipulate the control valve between an open state and a closed state in response to the pulsed control signal.

In some aspects, the disclosure relates to an aspiration control system, wherein the open state exposes the vacuum line to atmosphere and causes a decrease in the vacuum level within the vacuum line.

In some aspects, the disclosure relates to an aspiration control system, wherein the closed state seals the vacuum line from atmosphere and causes an increase in the vacuum level within the vacuum line.

In some aspects, the disclosure relates to an aspiration control system, wherein the control valve is a proportional flow valve.

In some aspects, the disclosure relates to an aspiration control system, wherein the vacuum regulator is configured to manipulate the proportional flow valve between an open state, a closed state, and at least one intermediate state between the open state and the closed state in response to the pulsed control signal.

In some aspects, the disclosure relates to an aspiration control system, wherein the sensor is arranged along the aspiration tubing between the receptacle and the aspiration catheter.

In some aspects, the disclosure relates to an aspiration control system, wherein the sensor is an ultrasonic flow sensor.

In some aspects, the disclosure relates to an aspiration control system, further including a pinch valve configured to fluidically couple to the vacuum source.

In some aspects, the disclosure relates to an aspiration control system, wherein the controller is operably coupled to the pinch valve, wherein the controller is further configured to: receive a state of the vacuum source, and transmit a pinch valve control signal to the pinch valve based on the state of the vacuum source, the pinch valve control signal being configured to manipulate a position of the pinch valve.

In some aspects, the disclosure relates to a computer-implemented method for controlling an aspiration system, wherein the aspiration system includes an aspiration catheter, a vacuum source, a receptacle, an aspiration tubing fluidically coupled to the receptacle and the aspiration catheter, a vacuum line fluidically coupled the receptacle and the vacuum source, a sensor configured to measure a flow parameter associated with a liquid within the aspiration tubing, and a vacuum regulator fluidically coupled to the vacuum line, the computer-implemented method including: receiving the flow parameter from the sensor; determining a flow condition based on the flow parameter; setting a characteristic of a pulsed control signal based on the flow condition; and transmitting the pulsed control signal to the vacuum regulator, the pulsed control signal being configured to control the vacuum regulator to adjust a vacuum level within the vacuum line.

In some aspects, the disclosure relates to an aspiration system including: an aspiration catheter; a thrombus retrieval device configured to extend through the aspiration catheter; a vacuum source; and the aspiration control system.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1A is a side section view of an implementation of the thrombectomy device having a single braided assembly in the collapsed configuration.

FIG. 1B is a side view showing the distal region of the thrombectomy device carrying the braided assembly of FIG. 1A. The braided assembly is shown in an expanded configuration.

FIG. 1C is a side view showing the distal region of the thrombectomy device of FIG. 1A. The braided assembly is not included in this view.

FIG. 1D is a cross sectional view of the implementation of FIG. 1A, taken along lines A-A of FIG. 1C.

FIG. 2 shows a side view of an implementation of a handle that can be used to control expansion and retraction of a braided assembly.

FIG. 3A is a side view of a distal region of an additional implementation of the thrombectomy device in an unexpanded configuration. The implementation has a braided assembly having multiple braided sections.

FIG. 3B is a side view of the distal region of the implementation of FIG. 3A in an expanded configuration.

FIG. 3C is a side view of the distal region of the thrombectomy device of FIG. 3A. The braided assembly is not included in this view.

FIG. 3D shows a cross sectional view taken along line 3D-3D of FIG. 3C.

FIG. 3E shows a cross sectional view taken along line 3E-3E of FIG. 3C.

FIG. 3F shows a cross sectional view taken along line 3F-3F of FIG. 3C.

FIG. 3G shows a cross sectional view taken along line 3G-3G of FIG. 3C.

FIG. 4 is a diagram of an aspiration control system for controlling blood loss during thrombus removal.

FIG. 5 is a flow chart of a method for controlling blood loss during thrombus removal.

FIG. 6 is a diagram of a computing device upon which aspects of the disclosed systems may be implemented.

FIG. 7 shows a proximal end of an aspiration catheter connected via an adapter to an aspiration tubing.

FIG. 8A shows an example pulsed control signal having pulses of 0.45 seconds length, where consecutive pulses are separated by 2.3 second delays and the total length of each cycle is 2.75 seconds.

FIG. 8B shows an example pulsed control signal having pulses of 0.8 seconds length, where consecutive pulses are separated by 2.3 second delays and the total length of each cycle is 3.1 seconds.

### DETAILED DESCRIPTION

The control systems and methods disclosed herein aim to reduce the amount of blood lost during procedures that utilize aspiration to remove thrombus from the vasculature. The control systems include, among other components, a controller that is used in conjunction with an aspiration catheter. The control system is equipped with a sensor in communication with controller. The controller monitors the flow rate of blood (e.g. measured by the sensor) being aspirated by a vacuum source (e.g. a pump) and communicates with a vacuum regulator to raise the vacuum if the flow rate gets to low (for example, when obstructed by a clot). The controller also communicates with the vacuum regulator to lower the vacuum if the flow rate gets too high. This lowering of the vacuum is a safety feature designed to prevent the system from aspirating blood instead of clot. Applying only the vacuum needed to remove the clot prevents unnecessary blood loss. Furthermore, damage to the vasculature is reduced because the amount of time the system applies full vacuum is minimized. The system can increase or decrease pressure gradually, or in a stepwise manner, to ensure the practitioner has sufficient time to respond to sudden changes in flow rate. The system can also include a variety of manual inputs to slow, pause, or stop the automated flow control processes.

The control system disclosed herein integrates flow and vacuum control, using flow rate to inform the desired vacuum level. As described herein, the controller generates and transmits pulsed control signals, the characteristics of which are based on flow conditions, to the vacuum regulator. Conventional systems do not offer this feature. Many prioritize improving aspiration efficiency over controlling blood loss. Furthermore, conventional systems often adjust vacuum level based on pressure sensors within the aspiration tubing. Because the disclosed system responds specifically to the parameter of interest—the amount of blood flowing through the catheter—it responds more accurately than pressure measurement-based systems. With this control system disclosed herein, the user can tune the flow rate, defining minimum and maximum allowed flow rates, and then automate control of the vacuum and flow rate according to those thresholds. This automation reduces the need for the practitioner to monitor and adjust the procedure for blood loss (though the practitioner is able to override the automation when necessary, via the aforementioned manual inputs). Advantageously, the control system minimizes the necessary procedure time by optimizing the aspiration process.

The aspiration control system can be used with a variety of catheters and vacuum sources (e.g. pumps) without changing the core aspiration control system. It can be provided with all of the necessary components, or can be used with components that are provided by the clinician. Furthermore, the controller, regulator, and pump can be used in the operating theater but fluidically isolated from the patient, negating the need for extensive sterilization. Flow manipulation occurs within the vacuum line instead of the aspiration lumen—a cleaner design than some conventional systems where valves directly manipulate blood flow within the aspiration lumen. In the disclosed aspiration control systems, components that do contact blood can be disposable, whereas fluidically isolated components can be reusable.

Example aspiration catheters and thrombectomy devices are described herein. This disclosure contemplates that the aspiration control systems of the present disclosure can be used with such aspiration catheters and/or thrombectomy device. It should be understood that the aspiration catheters and thrombectomy devices disclosed herein are only provided as examples. It should be understood that the aspiration control systems of the present disclosure can be used with different aspiration catheters and/or thrombectomy devices.

The thrombectomy devices disclosed herein remove a thrombus using a braided assembly that can be expanded to a diameter of the practitioner's choosing, enabling the practitioner to custom fit the device to the particular vessel and thrombus and during the procedure. Unlike conventional thrombectomy devices, the diameter of the disclosed braided assembly can be changed mid-procedure as needed. For example, the braided assembly can be opened to a wider diameter to apply more outward force against the thrombus should additional grip be needed for its removal. In some implementations, multiple braided assemblies can be used to address longer thrombi. Each braided assembly can be separately expanded, such that the individual assemblies have different diameters during the procedure.

The thrombectomy device disclosed herein is used to the remove a thrombus, clot, or plaque from the veins or arteries of the body. It includes an aspiration catheter and a retrieval device that extends through the lumen of the aspiration catheter. An expandable braided assembly extends over a distal region of the retrieval device, such that when the retrieval device exits the distal end of the aspiration catheter, the braided assembly is positioned outside of the aspiration catheter. An activation wire extends through the lumen of the retrieval device. The distal end of the activation wire exits the retrieval device at an exit point to connect to and control the expansion of a braided assembly. On the proximal end, the activation wire is attached to a tensioning element. Applying tension to the activation wire causes the braided assembly to expand to a diameter of the practitioner's choosing. For example, the practitioner may apply a first level of tension to deploy the braided assembly to a first, partially expanded configuration and then later decide to widen the diameter to the fully expanded configuration by applying a greater level of tension to the activation wire. The expanded braided assembly contacts the thrombus, clot, or plaque and is pulled proximally toward the aspiration catheter to assist in removal. Hereinafter the device and methods will be described as removing (or being configured to remove) a thrombus. However, it will be understood that the device can also be used to remove clots or plaques from the vasculature with no structural (or only slight structural) modifications. Various implementations of the thrombectomy catheter include a retrieval device with multiple braided assemblies, multiple activation wires, multiple braided sections of a single braided assembly, and retrieval devices with multiple lumens to, for example, enable use with a guidewire.

The following description of certain examples of the inventive concepts should not be used to limit the scope of the claims. Other examples, features, aspects, configurations, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation and may be applied to any aspect or implementation disclosed. It will understood that various changes and additional variations may be made and equivalents may be substituted for elements thereof without departing from the scope of the invention or the inventive concept thereof. Certain aspects and features of any given aspect may be translated to other aspects described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features, integers, characteristics, compounds, chemical moieties, or groups described in conjunction with a particular aspect, configuration, implementation or example of the invention are to be understood to be applicable to any other aspect, configuration, implementation, or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The invention is not restricted to the details of any foregoing aspects. The invention extends to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

The terms “proximal” and “distal” as used herein refer to regions of the aspiration tubing or thrombectomy device. “Proximal” means a region closest to the practitioner during a procedure, while “distal” means a region farther from the practitioner during a procedure.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

A vacuum is a pressure that is less than the local atmospheric pressure. As used herein, “vacuum pressure” indicates the relative pressure, i.e., the differential between ambient pressure and the absolute pressure applied by the vacuum. Thus, an increase in relative vacuum pressure is an increase in the difference between ambient pressure and the absolute vacuum pressure, which increases the force applied by the vacuum. Likewise, a decrease in relative vacuum pressure is a decrease in the difference between ambient pressure and the absolute vacuum pressure, which decreases the force applied by the vacuum.

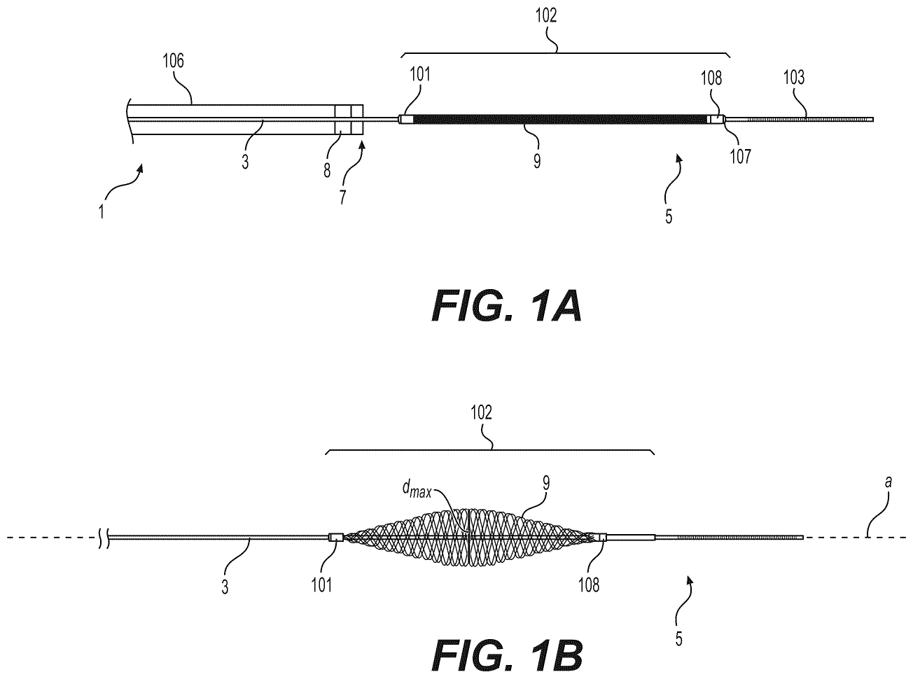

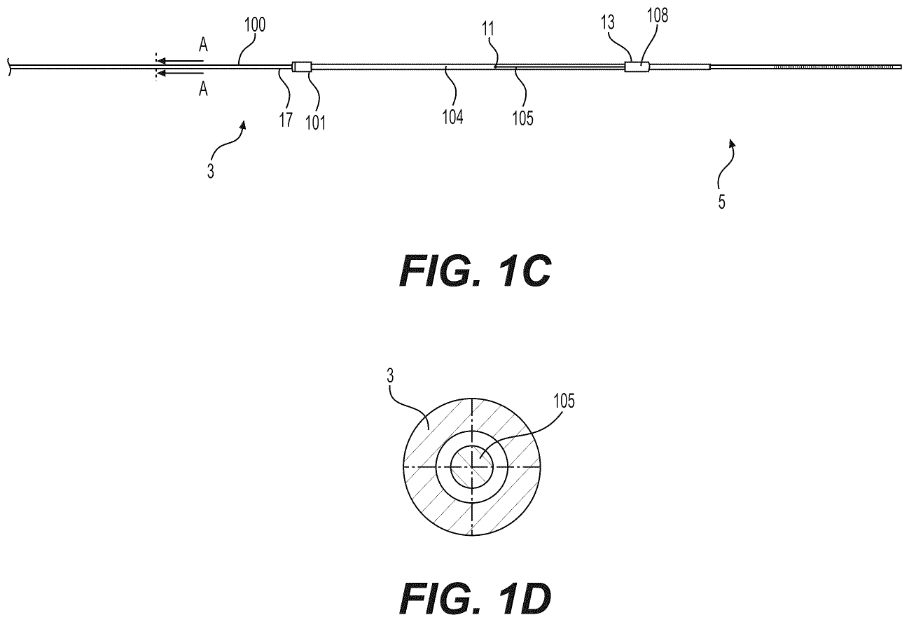

FIGS. 1A-1D show an implementation of the thrombectomy device 1. FIG. 1A shows the aspiration catheter 106, the retrieval device 3, a collapsed braided assembly 102, and a guidewire tip 103. The aspiration catheter 106 is an elongated tube with reinforced construction that allows a vacuum to be applied at the proximal end to pull clot and emboli out of the artery or vein without collapsing. The aspiration catheter 106 can be formed of a polymer material. The aspiration catheter 106 can include an imaging marker 8 (such as a fluorescent or radiopaque marker) for use in imaging the position of the catheter during a procedure. Thrombus retrieval device 3 extends through aspiration catheter 106. This disclosure contemplates that the control systems described herein are beneficial when using aspiration catheters having sizes greater than or equal to 10 F, e.g. 10 Fr, 11 Fr, 12 Fr, 13 Fr, 14 Fr, 15 Fr, 16 Fr, 17 Fr, 18 Fr, 19 Fr, 20 Fr, etc. As described in one example implementation below, the control system is used with aspiration catheters having sizes 14 Fr (small) and 16 Fr (large). Additionally, the control systems described herein may be beneficial when using aspiration catheters having sizes less than 10 F, e.g. 9 Fr, 8 Fr, or 7 Fr. The braided assembly 102 extends over a distal region 5 of the retrieval device 3, such that when the retrieval device 3 exits the distal end 7 of the aspiration catheter 106, the braided assembly 102 is positioned outside of the aspiration catheter 106. In the collapsed configuration, braided assembly 102 is sized and configured for insertion through the aspiration catheter 106 and into an artery or vein. Guidewire tip 103 extends distally from the distal end 107 of the retrieval device 3. The guidewire tip 103 can be flexible, shapeable, and steerable.

The braided assembly 102 is moveable from a collapsed to an expanded configuration. An example of a braided assembly 102 in an expanded configuration is shown in FIG. 1B, but the maximum diameter, dₘₐₓ, of the expanded braided assembly 102 can be changed to any value over a continuous range, from a fully collapsed diameter, to a partially expanded diameter, to a fully expanded diameter. The maximum diameter of the braided assembly, dₘₐₓ, is the widest point measured perpendicular to a longitudinal axis, a, extending through the center of the braided assembly 102. The braided assembly 102 can be sized and configured to disrupt and capture one or more clots, plaques, and/or thrombi and pull them toward the aspiration catheter 106 where they can be removed. The braided assembly 102 includes a braid 9, a slidable collar 108, and a fixed attachment point 101 where the braid 9 anchors to the retrieval device 3. The braid 9 may be attached directly to the retrieval device 3 at attachment point 101, or the braid 9 may be attached indirectly to the retrieval device 3 at attachment point 101. In some implementations, the fixed attachment point 101 is a fixed collar that extends around the retrieval device 3, and the braid is welded, bonded, or otherwise adhered to the fixed collar. Regardless, at the fixed attachment point 101, the braid 9 does not move longitudinally relative to the retrieval device 3.

The opposite end of braid 9 is welded, bonded, or otherwise adhered to slidable collar 108. In the implementations shown, the slidable collar 108 is slidably connected to the retrieval device 3 by virtue of its annular shape, which extends circumferentially around the retrieval device 3. The slidable collar 108 slides longitudinally along the retrieval device 3 as braid 9 is expanded and collapsed. The slidable collar 108 can be positioned distally to the fixed attachment point 101 (a distal position), as shown in FIGS. 1A-1C, or the slidable collar 108 can be positioned proximally to the fixed attachment point 101 (a proximal position). In some implementations, slidable collar 108 or fixed attachment point 101 can include a marker that can be viewed using imaging modalities during a procedure. For example, the slidable collar 108 or fixed attachment point 101 can include a fluorescent or radiopaque label.

The braid 9 is composed of multiple strands of wire. The braid 9 takes an elliptical or a spindle shape when expanded, having a maximum diameter dₘₐₓ at or near the center of the braid 9 and narrowing as the braid approaches the fixed attachment point 101 and the slidable collar 108. The wires are formed of a shape memory material such as, but not limited to, shape memory polymers or shape memory metals (e.g., nitinol). The braid 9 has a baseline shape memory of the collapsed configuration, which forms a cylindrical structure around the retrieval device 3, as shown in FIG. 1A. In the activated, expanded configuration, the braid 9 has a tendency to relax toward the collapsed configuration.

When the practitioner is pulling a thrombus or plaque proximally toward aspiration catheter 106 using braided assembly 102, the braid 9 encounters distally oriented drag forces that are strongest along the widest portions (for example, the central region of the braid adjacent dₘₐₓ). These drag forces resist the proximally oriented pulling force exerted by the practitioner. The distal end of braid 9 at slidable collar 108 will encounter less drag force while being pulled proximally because the radial force it exerts on the radially adjacent vasculature or thrombus is small, negligible, or non-existent. If the braid is not properly designed, the slidable collar 108 and distal end of the braid 9 will invert into the wider, central regions of the braid 9. Inversion during the procedure can be prevented by optimizing factors such as the pic count (crosses per inch), the wire diameter, the number of wires, and the ply of the braid (sets of overlapping braids). Higher pic counts increase flexibility, while lower pic counts increase longitudinal stiffness. Likewise, a braid with more than one ply (multiple sets of braids nested within each other), will be stiffer than a single-ply braid. Braids can be one-ply, two-ply, three-ply, or more. Braids with more wires will be stiffer than those with fewer wires, and braids with wider diameter wires will be stiffer than those with narrow diameter wires. Wires of varying diameters can be used within the same braid 9.

The design of the braided assemblies 102 disclosed herein may vary based on whether the device 1 is intended for an arterial procedure or for a venous procedure, since the procedure site will be wider in a venous setting. For example, a braid 9 designed for a venous application may have a dₘₐₓ of from about 0.8 inches to 1.2 inches, including about 0.8 inches, about 0.9 inches, about 1.0 inch, about 1.1 inches, and about 1.2 inches. For venous applications, a braid 9 may have a wire diameter range from about 0.005 inches to about 0.02 inches, including 0.005 inches, 0.0075 inches, 0.01 inches, 0.0125 inches, 0.015 inches, 0.0175 inches, and 0.02 inches. Different wires of the braid 9 may have different diameters, or they may have the same diameter. In some venous implementations, the diameters of the wires of the braid 9 are 0.01 inches, 0.0125 inches, and/or 0.015 inches. Two-ply braids can utilize smaller wire diameters without sacrificing the radial force that can be applied. The pic count can be from 2 to 6 for venous applications. In some implementations used in venous applications, the pic count is 3, 4, or 5. The number of wires per braid for a venous application can be anywhere from 8 to 40, including 8, 16, 24, 32, and 40.

For arterial applications, the braid 9 can have dₘₐₓ of from about 0.1 inches to about 0.4, including about 0.1 inches, about 0.12 inches, about 0.14 inches, about 0.18 inches, about 0.2 inches, about 0.22 inches, about 0.24 inches, about 0.28 inches, about 0.3 inches, about 0.32 inches, about 0.34 inches, about 0.36 inches, about 0.38 inches and about 0.4 inches. For example, the braid 9 can have a dₘₐₓ of about 0.28 inches, 0.3 inches, or 0.31 inches. The diameter of the wires of the braid 9 for an arterial application can range from about 0.001 inches to about 0.007 inches, including about 0.001 inches, about 0.002 inches, about 0.003 inches, about 0.004 inches, about 0.005 inches, about 0.006 inches, and about 0.007 inches. Different wires of the braid 9 may have different diameters, or they may have the same diameter. In some arterial implementations, the diameters of the wires of braid 9 are 0.003 inches, 0.004 inches and/or 0.005 inches. Two-ply braids can utilize smaller wire diameters without sacrificing the radial force that can be applied. The pic count can be from 5 to 30 for arterial applications, including a pic count of 5, 6, 7, 8, 9, 10, 11, 12, 13, 14, 15, 16, 117, 18, 19, 20, 21, 22, 23, 24, 25, 26, 27, 28, 29 and 30. In some implementations used in arterial applications, the pic count is 10, 12, or 15. The number of wires per braid 9 for an arterial application can be anywhere from 8 to 54, including 8, 16, 24, 32, 40, 48, and 54. In some implementations, the number of wires per braid 9 for an arterial application is 26, 24, or 30.

The activation wire 105 extends through the lumen of the retrieval device 3, exits the retrieval device 3 at exit point 11, and extends distally along the exterior surface of the retrieval device 3. The distal end 13 of the activation wire 105 is attached to slidable collar 108. As such, the activation wire 105 is able to control the expansion and collapse of the braid 9 via the slidable collar 108. The distance between exit point 11 and slidable collar 108 affects the length that the slidable collar can be pulled along retrieval device 3 to open the braided assembly 102. If it is too close to slidable collar, the braided assembly 102 will not be able to open fully. As such, exit point 11 should be positioned proximally far enough from the unexpanded position of slidable collar 108 to enable the braided assembly 102 to open to its maximum outer diameter. FIG. 1C shows the implementation of FIGS. 1A and 1B without braid 9 to facilitate viewing the activation wire 105 and the activation wire exit point 11. FIG. 1D is a cross sectional view of activation wire 105 in retrieval device 3, taken at line A-A of FIG. 1C. The internal positioning of the proximal regions of the activation wire 105 (within retrieval device 3) is advantageous in that no friction or bulk is added by the system that controls expansion of the braided assembly 102.

The proximal region of activation wire 105 (not shown) may be tensioned and released to control the expansion and collapse of the braided assembly 102 via movement of slidable collar 108. Under tension, the activation wire 105 moves proximally within the lumen of the retrieval device 3 as it translates the tension from the proximal region of the activation wire 105 to the braided assembly 102. In implementations where the slidable collar 108 is in the distal position (as shown), the exit point 11 of the activation wire is located proximally to the slidable collar 108. The exit point 11 can be, for example, a portal in the sidewall of retrieval device 3. Use of a slidable collar 108 to expand the braided assembly 102 is advantageous because the distal end of the braided assembly 102 can be moved while the distal region 5 of the retrieval device 3 maintains a constant position within the vasculature. Maintaining a constant position of the distal region 5 of retrieval device 3 is advantageous because sliding proximal/distal movement of the distal region 5 within the vessel can result in vessel damage or perforation.

In implementations where the slidable collar 108 is in the proximal position relative to the fixed attachment point (not shown), the activation wire 105 extends distally past the slidable collar 108 inside the retrieval device 3, exits the retrieval device 3 at exit point 11, then doubles back and extends along the exterior surface of the retrieval device 3 to attach to the proximally located slidable collar 108. The exit point 11 can be a portal in the sidewall of the retrieval device as described above, or the exit point 11 can be the distal end 107 of the retrieval device 3.

Retrieval device 3 can include a proximal hypotube 100 and a distal support tube 104, as shown in FIG. 1C. In some implementations, the hypotube 100 extends through the support tube 104. However, the distal region can be made more flexible by attaching the proximal end of the distal support tube 104 to the distal end 17 of the proximal hypotube 100 (for example, by adhesive bonding, heat bonding, or welding processes). The fixed attachment point 101 of the braided assembly 102 can be located on distal support tube 104 and the slidable collar 108 can extend around the distal support tube 104, such that the braided assembly 102 is positioned over and around the distal support tube 104. The braided assembly 102 can alternatively be positioned only partially over the distal support tube (i.e., one of the fixed attachment point 101 or the slidable collar 108 is attached to the proximal hypotube 100, and the other of the fixed attachment point or the slidable collar 108 is attached to the distal support tube 104). In some implementations, the support tube 104 serves to increase the overall diameter of the retrieval device 3, for example, to accommodate a larger diameter braid and to encapsulate the guidewire tip 103. The distal support tube 104 can also provide a lower friction surface for movement of the slidable collar 108 than the proximal hypotube 100 would provide.

In some implementations, distal support tube 104 has greater flexibility than the proximal hypotube 100. For example, the distal support tube 104 can be made of a polymer material, while the proximal hypotube 100 is made of a more rigid metal material. In some implementations, the proximal hypotube 100 is constructed from metal hypodermic needle tubing. The hypotube 100 can be up to 50 times stiffer than the support tube 104. There are several advantages to having a distal support tube 104 with greater flexibility than proximal hypotube 100. The greater flexibility of the support tube 104 enables a gradual transition in flexibility between the hypotube 100 and the guidewire tip 103. In some scenarios, the greater flexibility of the distal support tube 104 can facilitate movement of the braided assembly 102 through a tortuous thrombus. The greater flexibility can promote kink resistance. The greater flexibility of the distal support tube 104 can also facilitate the introduction of a portal or exit point 11 during the production of the device. The higher rigidity of the hypotube 100 (as compared to support tube 104) is important because it allows the retrieval device 3 to be pushed through the vasculature. The rigidity of hypotube 100 also helps to ensure that the braided assembly 102 can be pushed through a thrombus or plaque.

On the proximal end, the activation wire 105 can be attached to a tensioning element (not shown) that allows the activation wire 105 to be moved forward or retracted backward within the retrieval device 3. Applying tension to the activation wire 105 causes the slidable collar 108 to move and causes the braided assembly 102 to expand to a diameter of the practitioner's choosing. Similarly, releasing tension on the activation wire 105 allows the braided assembly 102 to relax into the collapsed, baseline configuration.

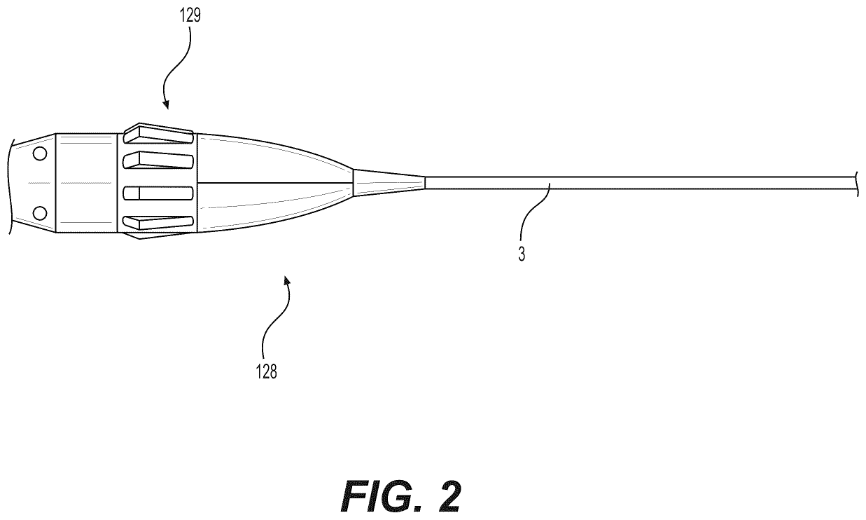

In some implementations, such as the one shown in FIG. 2, the device includes a proximal handle 128. The handle 128 is coupled to a proximal end of retrieval device 3. The tensioning element is a knob 129 that is coupled to the proximal end of activation wire 105 on the inside of the handle. Actuation of the knob 129 in one direction causes the activation wire 105 to be tensioned (expanding the braided assembly), and actuation of the knob 129 in the opposite direction releases tension on the activation wire 105 (collapsing the braided assembly). In other implementations, the tensioning element can include a slider, racheting mechanism, or lever.

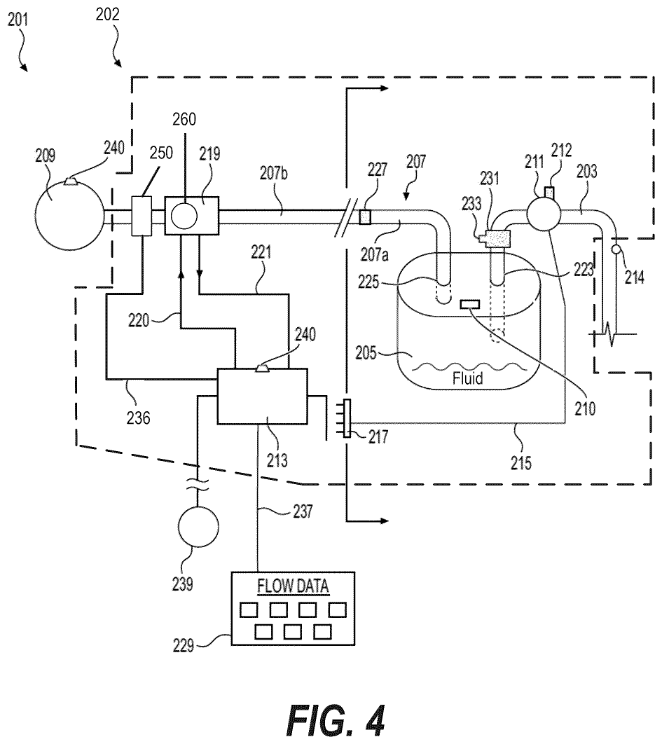

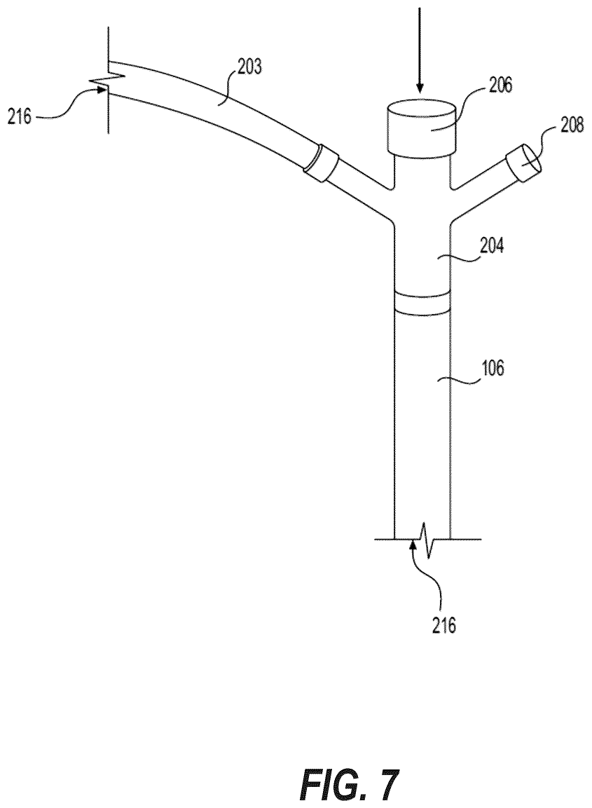

In some implementations, aspiration catheter 106 terminates in an adapter, such as adapter 204 shown in FIG. 7. The adapter 204 facilitates the connection of aspiration tubing 203, which can be connected to a vacuum system such as shown in FIG. 4 for removal of the clot or emboli. In some exemplary procedures, the distal end of retrieval device 3 is inserted axially through into the proximal end of adapter 204 (where shown using the arrow in FIG. 7). The retrieval device 3 is slid through the adapter 204 and subsequently through the aspiration catheter 106 until handle 128 abuts the proximal end of adapter 204.

Conventional thrombectomy devices utilize shape memory elements with a baseline expanded configuration. These conventional devices risk inadvertent overexpansion and damage to the vessel. Furthermore, conventional devices are often restrained by a bulky overlying sheath, which is pulled back to allow the device to self-expand.

Advantageously, using a device with a shape memory of the collapsed position reduces the risk of overexpansion and injury during self-expansion. Self-collapse also allows the device to be restrained using the low-profile activation wire system described herein. An additional advantage is the ability to expand the braided assembly to various diameters to precisely custom fit the size of the vessel. This can be especially useful if the size of the vessel is different than originally anticipated. The level of grip between the braid 9 and the surrounding thrombus can also be customized as needed by applying different levels of tension to the activation wire 105. For example, the practitioner may apply a first level of tension to deploy the braided assembly 102 to a first expanded outer diameter to contact the thrombus. If the force between the thrombus and the braid 9 is not enough to pull the thrombus toward the aspiration catheter 106, the practitioner can widen the braid 9 to a second expanded outer diameter by applying a greater second level of tension to the activation wire 105. This widened diameter provides a greater contact force between the thrombus and the braid 9, such that the thrombus can be more easily pulled toward aspiration catheter 106.

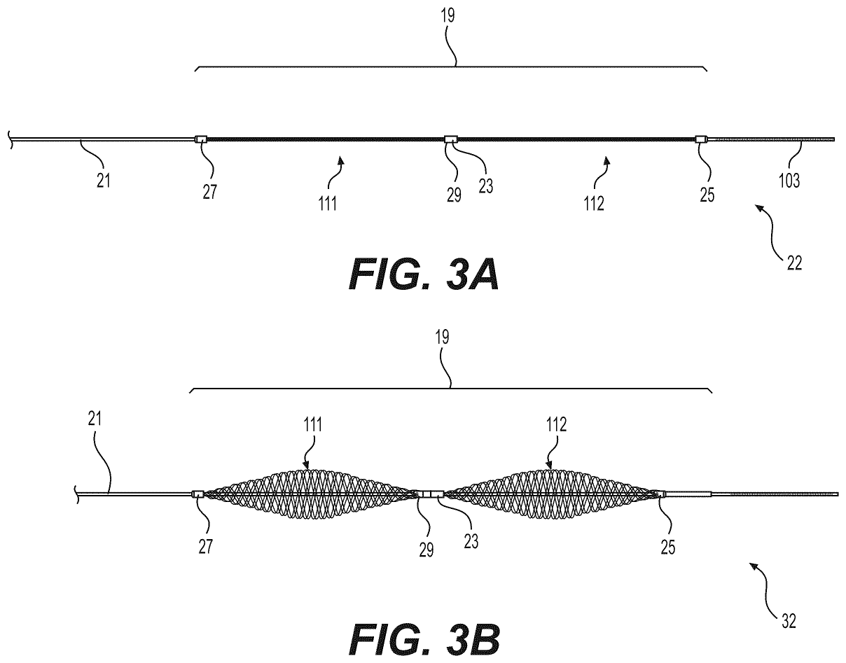

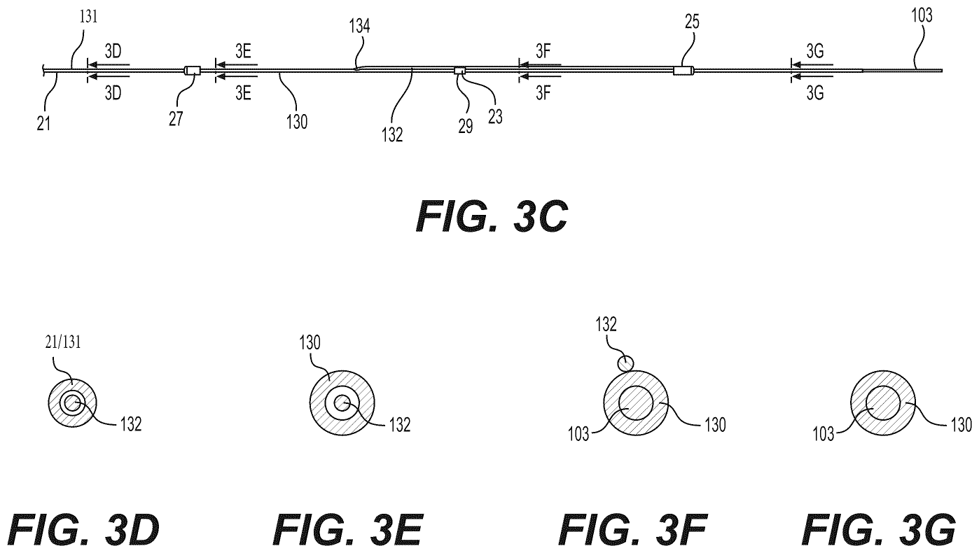

FIGS. 3A-3D show an additional implementation of a thrombectomy device having a braided assembly 19 with multiple braided sections 111, 112. The elongated nature of this implementation facilitates the capture and retrieval of long thrombi. As shown in FIG. 3A and FIG. 3B, ach of the braided sections 111, 112 is attached to and extends around the distal region 22 of retrieval device 21. The braided assembly 19 includes multiple sliding collars 23, 25 and a fixed attachment point 27. Proximal braided section 111 is attached to and extends between the fixed attachment point 27 and the proximal slidable collar 23, where it is welded, bonded, or otherwise adhered at a central sliding attachment point 29. Distal braided section 112 is attached to and extends between the proximal slidable collar 23 and the distal slidable collar 25. In some implementations, the braided sections are formed by constraining one larger braid with the proximal slidable collar 23. In other implementations, each braided section is formed from a separate braid (such that each of the proximal and distal braided assemblies are separately fixedly attached to proximal slidable collar 23). In some implementations, the slidable collars 23, 25 can be positioned distally to the fixed attachment point 27, as illustrated in FIG. 3A. In other implementations, the slidable collars can be positioned proximally to the fixed attachment point (not shown). Though illustrated with two braided sections 111, 112, other implementations of the braided assembly 19 could include more than two braided sections and more than two slidable collars.

FIG. 3C shows the thrombectomy device of FIGS. 3A and 3B without the braided assembly 19. Retrieval device 21 has a hypotube 131 fixedly attached to a support tube 130. A single activation wire 132 extends through hypotube 131 and support tube 130 to an exit point 134 positioned on the support tube 130. From there, it travels along the outer surface of support tube 130, running beneath proximal sliding collar 23 to attach to distal sliding collar 25. Cross sectional views shown in FIG. 3D, FIG. 3E, FIG. 3F, and FIG. 3G show the radial position of activation wire 132 with respect to hypotube 131, the support tube 130, and the guidewire tip 103 at various axial locations along the thrombectomy device shown in FIG. 3C. The activation wire 132 is utilized to control expansion of the braided assembly via connection to the distal sliding collar 25. In other implementations, the activation wire 132 can be attached to the proximal sliding collar 23. Proximal movement of the proximal slidable collar 23 or the distal slidable collar 25 by the activation wire generates a force on the other of the two slidable collars, such that the two braided sections 111, 112 are expanded (or partially expanded) in unison. As described above, the braids are formed of a shape memory material with a bias toward the collapsed configuration, so that tensioning the activation wire enables multiple levels of expansion.

The aspiration control systems and methods disclosed herein are configured to control blood loss during thrombus removal. An example aspiration system 201 is shown in FIG. 4. The aspiration system 201 can include a control system 202 (also referred to herein as an “aspiration control system”), a vacuum source 209, a power source 239, and/or a user interface 229. Additionally, the aspiration system 201 can include an aspiration catheter and a thrombus retrieval device. Example catheters and thrombus retrieval devices are described above with regard to FIGS. 1-3G.

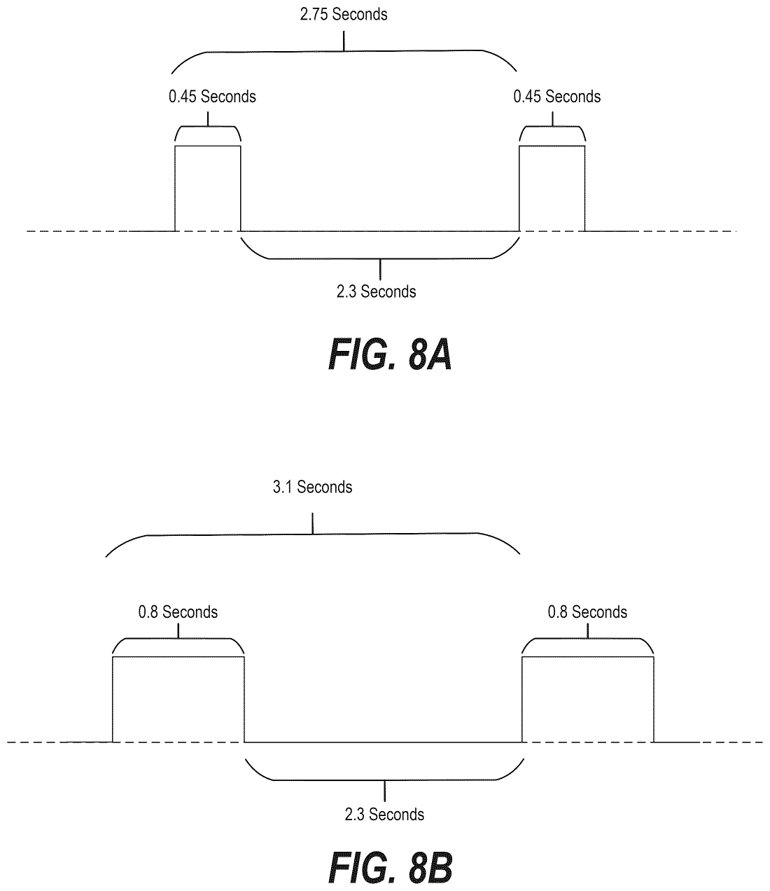

In some implementations described herein, the control system 202 can include a receptacle (sometimes referred to as a canister) 205, an aspiration tubing 203, a vacuum line 207, a sensor 211 configured to measure a flow parameter associated with a liquid within the aspiration tubing 203, a vacuum regulator 219 fluidically coupled to the vacuum line 207, and a controller 213. The aspiration tubing 203 is configured to fluidically couple the receptacle 205 to an aspiration catheter (not shown in FIG. 4). The vacuum line 207 is configured to fluidically couple the receptacle to a vacuum source 209. Additionally, the controller 213 is configured to: receive the flow parameter from the sensor 211, determine a flow condition based on the flow parameter, set a characteristic of a pulsed control signal based on the flow condition, and transmit the pulsed control signal to the vacuum regulator 219, the pulsed control signal being configured to control the vacuum regulator 219 to adjust a vacuum level within the vacuum line 207. Example pulsed control signals are illustrated in FIGS. 8A-8B. Optionally, as described herein, the control system 202 employed as part of the aspiration system 201, which can include, but is not limited to, an aspiration catheter, a thrombus retrieval device, and a vacuum source.

Referring to FIG. 4, in some implementations, the control system 202 includes an aspiration tubing 203, which extends from an adapter (see e.g., adapter 204 shown in FIG. 7). An aspiration lumen 216 extends from (and includes) the lumen of an aspiration catheter and the lumen of the aspiration tubing 203 leading to the receptacle 205. The receptacle or canister is a container for holding liquid. An example aspiration catheter 106 is shown in FIG. 1A. The aspiration tubing 203 is coupled to a receptacle 205 for collecting liquid aspirated by the aspiration catheter. In other implementations, aspiration catheter can be directly coupled to receptacle 205 for collecting liquid aspirated by the aspiration catheter. The aspiration tubing 203 (or aspiration catheter) creates a fluidic seal with the receptacle 205, and a vacuum line 207 is also fluidically coupled to and sealed with receptacle 205. The vacuum line 207 is fluidically coupled to a vacuum source 209.

The aspiration tubing 203 is coupled to one or more sensors 211. A sensor 211 is configured to measure a flow parameter associated with a liquid within the lumen of the aspiration tubing 203 (such as the flow rate, for example). The sensor 211 is operably connected to a controller 213, for example, via wiring 215 to 4-pin connector 217 (or any suitable connector). The controller 213 is also operably coupled to a vacuum regulator 219, for example, via wiring 220, and vacuum regulator 219 is coupled to vacuum line 207. The vacuum regulator 219 is configured to adjust the vacuum pressure within the vacuum line 207. In some implementations of the present disclosure, the sensor 211 is arranged along the aspiration tubing 203 between the receptacle 205 and the aspiration catheter.

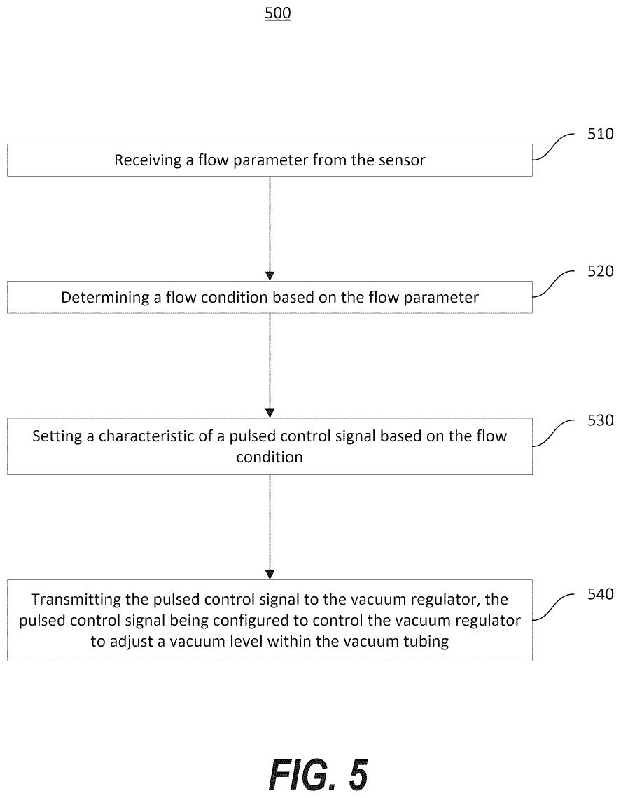

The vacuum regulator 219 optionally includes a control valve 260. In some implementations, the vacuum regulator 219 is configured to manipulate the control valve 260 between an open state and a closed state in response to a pulsed control signal generated by the controller 213. Additional description of the controller 213 and pulsed control signal is provided with reference to FIG. 5.

Optionally, the control valve 260 can be configured so that the open state exposes the vacuum line 207 the atmosphere. Exposing the vacuum line 207 to atmosphere can cause a decrease in vacuum level within the vacuum line 207. As used herein, a “decrease in the vacuum level” refers to an increase in pressure. Alternatively or additionally, the control valve 260 can be configured so that the closed state seals the vacuum line from atmosphere and causes an increase in the vacuum level within the vacuum line 207. As used herein, an “increase in the vacuum level” refers to a decrease in pressure.

In some implementations of the present disclosure, the control valve 260 is a proportional flow valve. Optionally, the vacuum regulator 219 can be configured to manipulate the proportional flow valve between an open state, a closed state, and at least one intermediate state between the open state and the closed state in response to the pulsed control signal.

In some implementations of the present disclosure, the system can further include a pinch valve 250 fluidically coupled to the vacuum source 209, as shown in FIG. 4. Optionally the controller 213 can be operably coupled to the pinch valve 250 (e.g., by wiring 236). The controller 213 can be configured to receive a state of the vacuum source, and transmit a pinch valve control signal to the pinch valve based on the state of the vacuum source. The pinch valve control signal can be configured to manipulate a position of the pinch valve 250. Non-limiting examples of pinch valve positions include open, closed, and/or one or more intermediate states between open and closed.

In some implementations of the present disclosure, the pinch valve 250 can be controlled by the controller 213 so that the pinch valve 250 is closed when vacuum is not being applied to the vacuum line 207 (i.e., “burst is off”). Closing the pinch valve 250 can prevent fluid flow from the patient caused by gravity and/or prevent fluid flow from the patient caused by the patient's blood pressure. Alternatively or additionally, closing the pinch valve 250 can prevent air from entering the aspiration tubing 203.

When the controller 213 receives the flow parameter from sensor 211, it compares the flow parameter to a desired target range and sends an automatic control signal to the vacuum regulator 219, based on the comparison of the flow parameter to the target range. As described herein, the controller 213 generates and transmits pulsed control signals, the characteristics of which are based on flow conditions, to the vacuum regulator 219. The automatic control signal causes the vacuum regulator 219 to adjust the vacuum pressure within vacuum line 207. For example, the automatic control signal can cause the regulator 219 to reduce the vacuum pressure upon a determination that the flow parameter is above an upper limit of the target range, or to decrease the vacuum pressure upon a determination that the flow parameter is below a lower limit of the target range. The controller 213 and the vacuum regulator 219 can optionally communicate in a feedback loop to regulate the vacuum pressure, wherein the controller 213 communicates a control signal to the vacuum regulator 219 and the vacuum regulator 219 communicates the current vacuum pressure back to the controller 213. In one example, communication from the vacuum regulator 219 to the controller 213 can be accomplished via wiring 221. These components and operations will be discussed in greater detail, below.

As described above, the vacuum line 207 is configured to fluidically couple the receptacle 205 to a vacuum source 209. In some implementations, the vacuum line 207 extends a relatively short distance into the receptacle 205 such that the vacuum line 207 is incapable of picking up liquid. This provides more volume in the vacuum line 207 for liquid collection. For example, as shown in FIG. 4, the vacuum line 207 does not extend as far as the intake port 223 into the receptacle 205. The vacuum source 209 can be a standard medical suction pump conventionally used in hospitals, outpatient surgery centers, and clinical practices. As one example, the vacuum source 209 can be a Basic Suction Pump (Medela AG, Baar, Switzerland), which has piston-cylinder based drive unit and mechanical overflow to protect the pump. The Medela Basic Suction pump has a max flow rate of 300 mL/min and a maximum vacuum of 90 kPa below ambient pressure. However, the make or model of vacuum source 209 is not limited—it can be any type available in clinical settings. The vacuum source 209 can be provided by the user, or it can be provided as part of a kit with some or all of the other components shown in FIG. 4. It should be understood that a pump is provided only as an example vacuum source 209. In some implementations, a wall vacuum (not shown) can be used as a vacuum source. As used herein, a “wall vacuum” refers to a centralized vacuum source that can provide vacuum to ports located in the wall of a building (e.g., a hospital or surgical center).

The aspiration tubing 203 is coupled to the receptacle 205 at an intake port 223, and the vacuum line 207 is coupled to the receptacle 205 at the vacuum port 225. The aspiration tubing 203, receptacle 205, and vacuum line 207 can be any make or model. The sizes and materials can be adapted to suit the needs of the particular procedure. The components shown on the right side of the arrows in FIG. 4, including the aspiration tubing 203, the flow sensor 211, the receptacle 205 and the vacuum line 207 can all optionally be disposable to reduce the need for sterilization between procedures. A connector 227 can be placed on the vacuum line 207 to enable connection of the disposal portion 207a of the vacuum line 207 with a reusable portion 207b of the vacuum line 207.

As discussed above, vacuum regulator 219 communicates with controller 213 to control the pressure in vacuum line 207. In one example, the vacuum regulator is an ITV 2090. As one example, the vacuum regulator 219 can include a control valve 260 that opens the vacuum line 207 to the atmosphere, thus decreasing the vacuum pressure by venting the system. As another example, regulation of flow can be achieved by incorporating an adjustable flow valve, which may widen or narrow the vacuum line 207 to alter the flow capacity (i.e., direct flow control). Notably, handling flow adjustments on the vacuum side (i.e., to the left of the arrow in FIG. 4) reduces the need to clean the flow handling components. This is an advantage over systems with components that physically restrict blood flow within the aspiration tubing.

The flow sensor 211 can be in direct contact with the liquid in the aspiration tubing 203, or it can be configured for contactless sensing. The flow sensor 211 sends measurements to the controller 213, which determines the flow rate accordingly. For example, in some implementations, the flow sensor 211 is an ultrasonic flow sensor positioned within the aspiration tubing 203, optionally within the aspiration lumen 216. Alternatively, in some implementations, the flow sensor 211 is a paddle-wheel style sensor positioned within the aspiration lumen 216. A paddle-wheel style sensor sends turn frequency measurements to the controller 213, which determines the flow rate accordingly. Alternatively, the flow sensor 211 can be a laser-based sensor, an infrared sensor, or some other optical based sensor, measuring the flow parameters through the walls of aspiration tubing 203 without contacting the blood. These have the benefit of being reusable, but are typically costlier than a contact-based sensor. Yet still additional example flow sensors that can be used as the flow sensor 211 of the present disclosure include differential pressure flow sensors, thermal flow sensors, target type flow sensors, turbine flow sensors, and magnetic induction flow sensors. In some implementations, the control system 202 includes a single sensor 211 as shown in FIG. 4. Optionally, in some implementations of the present disclosure it is further possible to incorporate a plurality of sensors that measure at disparate points along a length of the aspiration tubing 203. It should be understood that when a plurality of sensors are used one or more individual sensors may measure different flow values and that processing (e.g. correction, aggregation, etc.) may be needed to determine flow rate. In some examples, an interfacing filter can be positioned upstream from the flow sensor 211 to filter clots before they reach the sensor.

Adapter 204 shown in FIG. 7 facilitates the entry of the retrieval device 3 through proximal port 206 and into the lumen of the aspiration catheter 106. Adapter 204 also provides a fluidic coupling between the aspiration catheter and control system 202 (via aspiration tubing 203). Infusion port 208 allows for the introduction of saline or other infusion fluids. Adapter 204 houses an airtight valve that enables retrieval device to be introduced through proximal port 206 without passing air into the adapter 204. However, the aspiration tubing 203 can include an air leak sensor 214, such as an optical sensor, to detect air passage in case of valve failure. In some implementations, the air leak sensor 214 is positioned proximal to the flow sensor 211 to detect air before it reaches the vacuum system. Alternatively, the air leak sensor 214 can be integral with the flow sensor 211.

The air leak sensor 214 can communicate a leak signal to the controller 213, the vacuum regulator 219, or directly to the vacuum source 209, at which point the controller 213, the vacuum regulator 219, or the vacuum source 209 slows or stops the vacuum flow while the air leak is addressed, reducing blood loss. In some implementations, there is a leak detection delay during initialization of the vacuum. The leak detection delay prevents the aspiration of the initial air in the tube—otherwise, the air leak sensor may detect it as a leak.

As shown in FIG. 4, the flow sensor 211 can be in communication with an indicator 212. The indicator 212 can be configured to inform the user of a characteristic of the flow parameter or flow rate. For example, the indicator 212 can be an LED light or an LED light panel. Optionally the indicator 212 can indicate the flow rate by varying brightness of a light. Alternatively or additionally, the indicator 212 can indicate the flow rate by flashing a light. This disclosure contemplates that variations in luminosity and/or flashing frequency can be used to indicate the flow rate through the aspiration tubing 203. The indicator could alternatively be audible, or it could be a display on a user interface 229. The indicator 212 is shown positioned on the flow sensor 211, but in operation could be positioned anywhere within the system. The flow sensor 211 can communicate directly with the indicator 212, or via the controller 213.

The system provide for one or more manual override options, which may communicate a complete shut-off of the vacuum or aspiration flow, a scaled diminishing of the vacuum or aspiration flow, a reactivation of vacuum or aspiration flow, or a scaled increasing of the vacuum or aspiration flow. The manual commands are accepted at one or more manual inputs, such as, but not limited to, clamps, switches, buttons, sliders, knobs, pedals or a digital user interface. In some examples, a clamp 231 can be coupled to the aspiration tubing 203 to enable the practitioner to manually stop the flow of blood through the aspiration tubing 203, overriding the automated system. Alternatively or additionally, a manual override switch or button can communicate with the vacuum regulator 219 to fully open the valve to atmospheric pressure, thereby releasing the pressure in the vacuum line 207. As an alternative to a binary switch or button that fully releases the vacuum, sliders, knobs, or the like can be used to diminish or increase the vacuum force gradually. As another alternative, a foot pedal (not shown) can be activated to shut down or slow the flow either at the clamp 231, the vacuum regulator 219, or the vacuum source 209 (or reactivate and increase the flow at any of those components). In some implementations, a manual override option can be provided at a user interface 229 (including manual or digital switches, sliders or knobs). Finally, the user has the option to turn the vacuum on and off at the vacuum source 209. In the implementations described herein, the vacuum source 209 is either ON or OFF. Additionally, vacuum strength is gradually adjusted using the vacuum regulator 219 as described below.

Triggering a manual override option can induce a first manual control signal to be sent from the manual input to the controller 213. The first manual control signal can cause the controller 213 to stop performing the step of comparing the flow parameter to the target range, or to stop the step of forming the automatic control signal, or to stop the step of sending the automatic control signal to the regulator. The controller 213 can instead send a second manual control signal to the vacuum regulator 219 based on the manual input (information from the first manual control signal). Alternatively, triggering a manual override option can send a manual control signal from the manual input directly to the vacuum regulator 219 to raise or lower the vacuum pressure in the vacuum line 207. Likewise, the manual input can be triggered to reactivate any or all of these processes.

In some implementations, the receptacle 205 can include a receptacle vent 210 in communication with the controller 213, the vacuum source 209, the vacuum regulator 219, or with one or more of the manual inputs. Any one of the above-listed manual override actions can trigger a receptacle venting signal to be sent to the receptacle vent 210, directly or via the controller 213. The vent receives the receptacle venting signal and opens to equilibrate the pressure in the receptacle 205 with atmospheric pressure. This action eliminates residual pressure in the receptacle 205 that might otherwise cause the suction of additional blood after the manual override action. In some aspects, the venting signal may be a manual control signal sent by the controller 213 or vacuum regulator 219 upon activation of one or more manual inputs (for example, a manual command to override the vacuum system).

The clamp 231 can be coupled to tubing between the aspiration tubing 203 and the receptacle 205 and can be activated by a switch 233 for ease of use. The switch 233 can have an open configuration and a closed configuration. In the closed configuration, the switch tells the clamp 231 to close off the flow to the receptacle 205. In the open configuration, the switch 233 tells the clamp 231 to open the aspiration tubing 203 and allow blood to flow into receptacle 205. However, closure of clamp 231 can cause vacuum pressure to build up in the vacuum line 207. Opening the clamp 231 can thus cause a sudden surge and suction of blood. To avoid this, the switch 233 can be in operable communication with the vacuum regulator 219. Alternatively or additionally, the switch 233 can be a manual switch that can be opened or closed by a user. The shifting of switch 233 to the open configuration sends a surge signal to the vacuum regulator 219, either directly or via controller 213 as shown in FIG. 4. The surge signal causes the vacuum regulator 219 to decrease the vacuum pressure in vacuum line 207, thereby reducing the surge of blood associated with opening the clamp 231. In some examples, the switch can be configured to cause a lowering of the vacuum pressure in the vacuum line 207 before releasing the clamp 231.

Some examples of the system include a user interface 229. The user interface 229 can optionally be a touch screen, or it can be in communication with a keyboard. Regardless, the user interface can be configured to accept manual input. The user interface 229 can be in communication with the controller 213 or with the vacuum regulator 219, and is configured to send manual control signals to the controller 213 or the vacuum regulator 219 responsive to the manual input. For example, the user can adjust the upper and lower limits of the target range and/or the time delay in triggering a vacuum change (for example, how long the flow rate is permitted to be at or above the upper limit before the vacuum pressure is decreased). The user interface can include a manual override button that allows the user to stop automated control and manually set the flow rate through the aspiration tubing 203. The user interface 229 can also include readouts to inform the user of the instantaneous flow rate through the aspiration tubing 203, and/or the total blood collected at receptacle 205.

In some implementations, the aspiration tubing 203 optionally includes a radiofrequency identification (RFID) key. Additionally, the aspiration system 201 can optionally include an RFID reader. Optionally, the RFID reader may be a component of the control system 202.

Communication between the components of control system 202 can take occur via any suitable communication link that facilitates data exchange between the components. Communication links can include wired, wireless, and optical links. Example communication links include, but are not limited to, a local area network (LAN), a wireless local area network (WLAN), a wide area network (WAN), a metropolitan area network (MAN), Ethernet, the internet, or any other wired, or wireless link such as WiFi, WiMax, 3G, 4G, or 5G. As shown in FIG. 4, the communication links can be formed of electrical or optical wires 215, 220, 221, and 237. However, the presence of some wires in the drawing is not intended to indicate that those communication links, or any communication links, within the system must be hard-wired. Any of the communication links disclosed herein can be wired or wireless in nature.

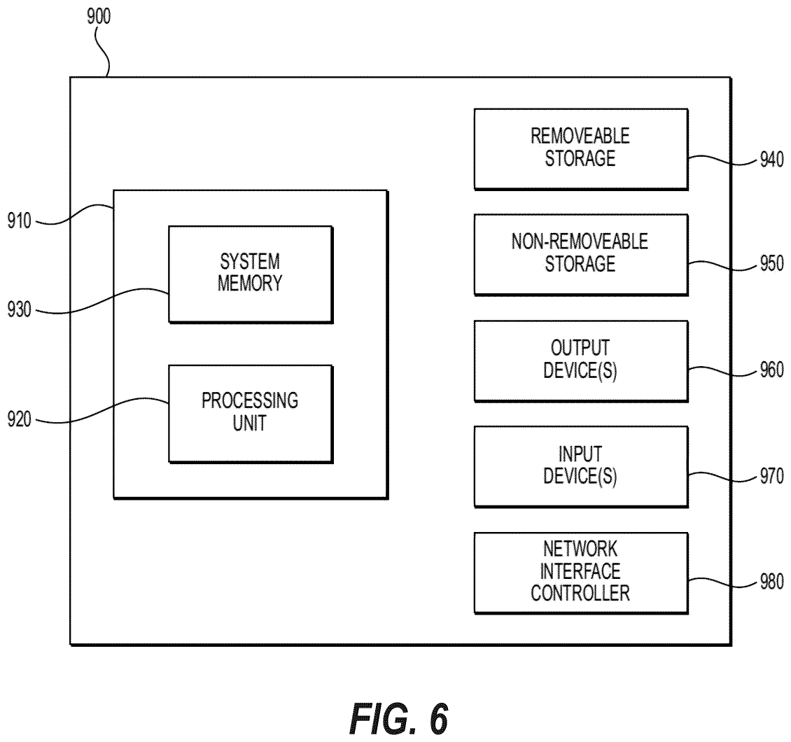

The controller 213 can include a computing device. For example, this disclosure contemplates that the controller 213 can include at least a processor and memory, i.e. a basic computer configuration described with regard to FIG. 6. Optionally, the controller 213 can advantageously be a microprocessor that operates independently of a larger computer system. For example, controller 213 can be a complex instruction set microprocessor, a reduced instruction set microprocessor, a superscalar processor, an application specific integrated circuit, or a digital signal multiprocessor. It should be understood that a microprocessor includes at least a processor and memory. The controller 213 is configured to measure frequency from the flow parameter received from sensor 211, and includes an analog to digital converter and a digital to analog converter. Power source 239 can be any suitable power source, and can be external to the controller 213 (such as a wall outlet) or internal to the controller 213 (such as a battery).

In some implementations, power sensors 240 can be included on the vacuum source 209 and/or the controller 213 in order to synchronize the delivery of power between the two components. That is, when one of the two components (vacuum source 209 or controller 213) is powered off, the power sensor 240 on the other of the two components detects the power change and is automatically powered off. Likewise, when one of the two components (vacuum source 209 or controller 213) is powered on, the other is also automatically powered on. Optionally, the vacuum source 209 includes an AC powered electric pump, and the power sensor 240 can be configured to sense the AC current draw of the pump. By sensing the AC current draw of the pump using a power sensor 240, the controller 213 can detect that the vacuum source 209 is generating vacuum. It should be understood that an AC powered electric pump is intended only as a non-limiting example, and that other implementations of the present disclosure can include different types of pumps.

With the basic structure of the control system 202 being thusly disclosed, a greater appreciation of the construction and benefits of may be gained from the following discussion of the operation of the control system 202 as shown in the flow chart at FIG. 5 and in reference to the diagram of FIG. 4. It is to be noted that this discussion is provided for illustrative purposes only. A method of controlling blood loss during thrombus removal is disclosed herein that can be implemented, for example, by the control system 202 shown in FIG. 5.

With reference to FIG. 5, an implementation of a method 500 is shown. The method 500 can be implemented using the controller 213 of the control system 202 described with reference to FIG. 4. This disclosure contemplates that the logical operations can be performed using a computing device such as the computing device shown in FIG. 6.

At step 510, the controller can receive a flow parameter from a sensor (e.g., the flow sensor 211 shown in FIG. 4).

At step 520, the controller can determine a flow condition based on the flow parameter.

In some implementations, a high flow condition and/or a low flow condition can be determined. In response to determining a high flow condition, the characteristic of the pulsed control signal can be set to a first value, and in response to determining a low flow condition, the characteristic of the pulsed control signal can be set to a second value, where the second value is different than the first value. As described below, the characteristic of the pulsed control signal can include, but is not limited to, pulse duration, frequency, and duty cycle.

Optionally, the flow condition can be determined by comparing the flow parameter to a threshold value. As non-limiting examples, the threshold value can be between about 70 mL/min and about 130 mL/min in some implementations of the present disclosure. For example, the threshold value can be about 70 mL/min, about 80 mL/min, about 90 mL/min, about 100 mL/min, about 110 mL/min, about 120 mL/min, or about 130 mL/min. Optionally, in some implementations of the present disclosure, the threshold value can be about 100 mL/min.

In some implementations of the present disclosure, the flow parameter received at step 510 can be greater than or equal to the threshold value in a high flow condition.

In some implementations of the present disclosure, the flow parameter received at step 510 can be less than the threshold value in a low flow condition.

At step 530 the controller can set a characteristic of a pulsed control signal based on the flow condition. As used herein, the “characteristic” of the pulsed control signal can refer to any of the following non-limiting example characteristics, alone or in combination: duration, amplitude, direction, waveform shape, hysteresis, duty cycle, phase, and frequency.

Optionally, the characteristic of the pulsed control signal is a pulse duration. Optionally, in response to determining a high flow condition, the pulse duration is set to a first value, and in response to determining a low flow condition, the pulse duration is set to a second value, where the second value is greater than the first value. As non-limiting examples, the first value can be about 0.45 seconds, and the second value can be about 0.80 seconds.

Alternatively or additionally, the characteristic of the pulsed control signal is a pulse frequency. Optionally, in response to determining a high flow condition, the pulse frequency is set to a first value, and in response to determining a low flow condition, the pulse frequency is set to a second value, where the first value is greater than the second value. As non-limiting examples, the first value can be about 22 bursts per minute, and the second value can be about 19 bursts per minute.

Alternatively or additionally, the characteristic of the pulsed control signal is a duty cycle. Optionally, in response to determining a high flow condition, the duty cycle is set to a first value, and in response to determining a low flow condition, the duty cycle is set to a second value, where the second value is greater than the first value. Optionally, the first value is about 20%, and the second value is about 25%.

At step 540, the controller can transmit the pulsed control signal to the vacuum regulator (e.g., the vacuum regulator 219 shown in FIG. 4). The pulsed control signal can be configured to control the vacuum regulator to adjust a vacuum level within the vacuum line (e.g., the vacuum line 207 shown in FIG. 4). Optionally, the vacuum regulator can be actuated between an open state and a closed state, where the open state exposes the vacuum line to the atmosphere. Exposing the vacuum line to atmosphere can cause a decrease in vacuum level within the vacuum line. As used herein, a “decrease in the vacuum level” refers to an increase in pressure. Alternatively or additionally, the vacuum regulator can be configured in a closed state, where the closed state seals the vacuum line from atmosphere and causes an increase in the vacuum level within the vacuum line. As used herein, an “increase in the vacuum level” refers to a decrease in pressure.

In one example implementation of the present disclosure, the control system can be configured to control the flow from a small catheter and/or a large catheter. As used herein, “large catheter” refers to a 16 Fr size catheter, and “small catheter” refers to a 14 Fr size catheter. Optionally, the pulsed control signals of the present disclosure can refer to “bursts” where the vacuum regulator is configured to cause fluid to flow from the patient. Example pulsed control signals are illustrated by FIGS. 8A-8B.

In the example, the open flow rate of the small catheter is 1000 ml in 32 seconds, or about 32 ml per second. The open flow rate of the large catheter is about 1000 ml in 24 seconds, or about 42 ml per second.

When either catheter is not in a clot, the controller detects a high flow condition and shortens the burst time to 0.45 seconds. When either catheter is in the clot, the controller detects a low flow condition, and the burst time is set to 0.8 seconds. Optionally, the flow can “ramp up” and/or “ramp down” during the burst, which results a reduced flow rate during bursts. As a non-limiting example, the flow rate during a burst can be about 50% of the steady state flow rate due to the ramp up and/or ramp down during the burst. Optionally, the time between bursts can be 2.3 seconds.

With the example small catheter, the flow rate when not in a clot can be calculated as follows: 32 ml/sec for 0.45 sec=14 ml/Burst×50%=7 ml/Burst. With the example small catheter, the flow rate when in the clot can be calculated as follows: 32 ml/sec for 0.8 sec=26 ml/Burst,×50%=13 ml/Burst.

With the example large catheter, the flow rate when not in the clot can be calculated as follows: 42 ml/sec for 0.45 sec=19 ml/Burst×50%=10 ml/Burst. With the example large catheter, the flow rate when in the clot can be calculated as follows: 42 ml/sec for 0.8 sec=34 ml/Burst,×50%=17 ml/Burst. Thus, the amount of fluid lost per burst can vary from 7 ml per burst with the small catheter to 17 ml per burst with the large catheter.

In a non-limiting example of a procedure, the system can be used for 1 minute. When the burst time is set to 0.8 seconds, the number of bursts in one minute can be calculated as 60 sec/(2.3 sec+0.8)=19 Bursts. When the burst time is set to 0.45 seconds, the number of bursts in one minute can be calculated as: 60 sec/(2.3 sec+0.45)=22 Bursts. FIG. 8A illustrates 0.45 second pulses, which can correspond to 0.45 second burst times. FIG. 8B illustrates 0.8 second pulses, which can correspond to 0.8 second burst times. The total time of each cycle in FIG. 8A is 2.75 seconds, and the total time of each cycle in FIG. 8B is 3.1 seconds.

Optionally, the maximum amount of fluid loss can be determined using the flow rate and burst time. In the large catheter with the highest flow described above, 22 bursts per minute, times 17 ml per burst, gives a maximum of 374 ml/minute of fluid flow. In practice, with the large catheter in the clot, the fluid flow is significantly reduced. In a study performed of an example implementation, the actual flow was less than 100 ml per minute.

It should be appreciated that the logical operations described herein with respect to the various figures may be implemented (1) as a sequence of computer implemented acts or program modules (i.e., software) running on a computing device (e.g., the computing device described in FIG. 6), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 6, an example computing device 900 upon which implementations of the invention may be implemented is illustrated. For example, the controller 213 may each be implemented as a computing device, such as computing device 900. It should be understood that the example computing device 900 is only one example of a suitable computing environment upon which implementations of the invention may be implemented. Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In an implementation, the computing device 900 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an implementation, virtualization software may be employed by the computing device 900 to provide the functionality of a number of servers that is not directly bound to the number of computers in the computing device 900. For example, virtualization software may provide twenty virtual servers on four physical computers. In an implementation, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third party provider. Some cloud computing environments may comprise cloud computing resources owned and operated by the enterprise as well as cloud computing resources hired and/or leased from a third party provider.

In its most basic configuration, computing device 900 typically includes at least one processing unit 920 and system memory 930. Depending on the exact configuration and type of computing device, system memory 930 may be volatile (such as random access memory (RAM)), non-volatile (such as read-only memory (ROM), flash memory, etc.), or some combination of the two. This most basic configuration is illustrated in FIG. 6 by dashed line 910. The processing unit 920 may be a standard programmable processor that performs arithmetic and logic operations necessary for operation of the computing device 900. While only one processing unit 920 is shown, multiple processors may be present. Thus, while instructions may be discussed as executed by a processor, the instructions may be executed simultaneously, serially, or otherwise executed by one or multiple processors. The computing device 900 may also include a bus or other communication mechanism for communicating information among various components of the computing device 900.

Computing device 900 may have additional features/functionality. For example, computing device 900 may include additional storage such as removable storage 940 and non-removable storage 950 including, but not limited to, magnetic or optical disks or tapes. Computing device 900 may also contain network connection(s) 980 that allow the device to communicate with other devices such as over the communication pathways described herein. The network connection(s) 980 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing device 900 may also have input device(s) 970 such as a keyboards, keypads, switches, dials, mice, track balls, touch screens, voice recognizers, card readers, paper tape readers, or other well-known input devices. Output device(s) 960 such as a printers, video monitors, liquid crystal displays (LCDs), touch screen displays, displays, speakers, etc. may also be included. The additional devices may be connected to the bus in order to facilitate communication of data among the components of the computing device 900. All these devices are well known in the art and need not be discussed at length here.

The processing unit 920 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 920 for execution. Example tangible, computer-readable media may include, but is not limited to, volatile media, non-volatile media, removable media and non-removable media implemented in any method or technology for storage of information such as computer readable instructions, data structures, program modules or other data. System memory 930, removable storage 940, and non-removable storage 950 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

In an example implementation, the processing unit 920 may execute program code stored in the system memory 930. For example, the bus may carry data to the system memory 930, from which the processing unit 920 receives and executes instructions. The data received by the system memory 930 may optionally be stored on the removable storage 940 or the non-removable storage 950 before or after execution by the processing unit 920.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

Implementations of the methods and systems may be described herein with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

### EXAMPLE

Conventional aspiration systems operate with the vacuum being controlled with a “foot valve” by the operating physician. This is not very precise and is a distraction to the primary job of guiding the catheter. The principle of this system is to automate the process. The reader is directed to the flow chart of FIG. 5 for reference.

The system is controlled by a microprocessor controller (MC). A simple device as compared to a conventional computer and can run “stand alone”. That means all it needs to run is powering it up. The program is written to be failsafe and straightforward. On power up the MC initializes and performs a test of the system and a checksum on the program. Any problem with this stops the process. The system uses a Vacuum Regulator (VR) module. It takes raw unregulated vacuum from a user supplied vacuum pump (VP). The VR can be set to deliver from full to almost zero vacuum by the MC while the VP is set to maximum vacuum.

Another component of the system is the Flow Sensor (FS). This device measures the amount of blood flow out of the catheter. The Flow Sensor can include any of the flow sensors 211 described with reference to FIG. 4. As also described with reference to FIG. 4, the Flow Sensor of the present example can include multiple sensors.

An ultrasonic flow sensor works well in this application and can accurately measure the low flow rates that might indicate the possibility of a blockage caused by a blood. A small tube from the catheter is placed in a cavity on the sensor and the velocity of the flow is determined. An electrical pulse train is produced that has a frequency proportional to the flow rate that is read by the MC. The sensor is completely isolated from any liquids and can be used repeatedly, whereas the rotational sensor would have to be disposed of after every procedure.

In this example, 100 mL/minute is the upper limit on the flow rate. After power on test is complete, the VR commands full vacuum. The flow can reach 100 mL/min quickly, and the MC detects that and quickly commands the VR to reduce vacuum and to control the flow by adjusting the vacuum to a midline flow rate of around 30 mL/min. Without the VR, blood flow out of the catheter would be excessive. Alternatively or additionally, the MC controller can use pulsed control signals to the VR to control the flow rate, as described above. The pulsed control signals can cause the VR to deliver bursts of full vacuum power separated by times between bursts with no vacuum power. By controlling the number of bursts, the burst times, and the time between bursts, the MC controller can control the flow to prevent excessive blood flow from the catheter. Optionally, the MC controller can be configured so that the number of bursts, burst times, and times between bursts can be based on the size of the catheter, so that the system prevents excessive blood flow when using catheters of different sizes. In this example, the pulsed flow rate can be more effective at loosening clots than a continuous flow without applying the bursts described with reference to this example.

The flow rate from 100 mL/min to 30 mL/min can be achieved in steps so that the clinician gets enough time to make necessary decisions/actions. Same delay time or stepped approach is taken for the application of full vacuum to the aspiration tubing when the flow rate falls below a lower limit of 10 ml/min (or the tip of the catheter engages a clot, in practice).

If, while the system is regulating to 30 mL/min, it drops below 10 mL/min, a blockage of the catheter has probably occurred. The system will attempt to “Clear” the blockage, usually caused by encountering a blood clot, by commanding full vacuum power from the VR. As in the beginning, when the flow hits 100 mL/min, the MC will command the VR will scale back and regulate to 30 mL/min.

The cycle repeats until the unit is shut off. Before complete shutdown, the MC commands the VR to completely release the vacuum.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

1. An aspiration control system comprising:
a receptacle;
an aspiration tubing configured to fluidically couple the receptacle to an aspiration catheter;
a vacuum line configured to fluidically couple the receptacle to a vacuum source;
a sensor configured to measure a flow parameter associated with a liquid within the aspiration tubing;
a vacuum regulator fluidically coupled to the vacuum line; and
a controller operably coupled to the sensor and the vacuum regulator, wherein the controller is configured to:
receive the flow parameter from the sensor,
determine a flow condition based on the flow parameter,
set a characteristic of a pulsed control signal based on the flow condition, and
transmit the pulsed control signal to the vacuum regulator, the pulsed control signal being configured to control the vacuum regulator to adjust a vacuum level within the vacuum line.

2. The aspiration control system of claim 1, wherein:
in response to determining a high flow condition, the characteristic of the pulsed control signal is set to a first value, and
in response to determining a low flow condition, the characteristic of the pulsed control signal is set to a second value, wherein the second value is different than the first value.

3. The aspiration control system of claim 1, wherein the characteristic of the pulsed control signal is a pulse duration.

4. The aspiration control system of claim 3, wherein:
in response to determining a high flow condition, the pulse duration is set to a first value, and
in response to determining a low flow condition, the pulse duration is set to a second value, wherein the second value is greater than the first value.

5. The aspiration control system of claim 4, wherein the first value is about 0.45 seconds, and the second value is about 0.80 seconds.

6. The aspiration control system of claim 1, wherein the characteristic of the pulsed control signal is a pulse frequency, and wherein in response to determining a high flow condition, the pulse frequency is set to a first value, and in response to determining a low flow condition, the pulse frequency is set to a second value, wherein the first value is greater than the second value, wherein the first value is about 22 bursts per minute, and the second value is about 19 bursts per minute.

7. (canceled)

8. (canceled)

9. The aspiration control system of claim 1, wherein the characteristic of the pulsed control signal is a duty cycle, and wherein:
in response to determining a high flow condition, the duty cycle is set to a first value, wherein the first value is about 20%, and
in response to determining a low flow condition, the duty cycle is set to a second value, wherein the second value is greater than the first value, and wherein the second value is about 25%.

10. (canceled)

11. (canceled)

12. The aspiration control system of claim 1, wherein the flow condition is determined by comparing the flow parameter to a threshold value, wherein the threshold value is between about 70 mL/min and about 130 mL/min, and wherein the flow parameter is greater than or equal to the threshold value in a high flow condition and wherein the flow parameter is less than the threshold value in a low flow condition.

13. (canceled)

14. (canceled)

15. (canceled)

16. (canceled)

17. The aspiration control system of claim 1, wherein the vacuum regulator comprises a control valve arranged along the vacuum line between the vacuum source and the receptacle.

18. (canceled)

19. The aspiration control system of claim 17, wherein the vacuum regulator is configured to manipulate the control valve between an open state and a closed state in response to the pulsed control signal.

20. The aspiration control system of claim 19, wherein the open state exposes the vacuum line to atmosphere and causes a decrease in the vacuum level within the vacuum line.

21. The aspiration control system of claim 19, wherein the closed state seals the vacuum line from atmosphere and causes an increase in the vacuum level within the vacuum line.

22. The aspiration control system of claim 17, wherein the control valve is a proportional flow valve.

23. The aspiration control system of claim 22, wherein the vacuum regulator is configured to manipulate the proportional flow valve between an open state, a closed state, and at least one intermediate state between the open state and the closed state in response to the pulsed control signal.

24. The aspiration control system of any one of claims 1, wherein the sensor is arranged along the aspiration tubing between the receptacle and the aspiration catheter.

25. The aspiration control system of claim 1, wherein the sensor is an ultrasonic flow sensor.

26. The aspiration control system of claim 1, further comprising a pinch valve configured to fluidically couple to the vacuum source.

27. The aspiration control system of claim 26, wherein the controller is operably coupled to the pinch valve, wherein the controller is further configured to:
receive a state of the vacuum source, and
transmit a pinch valve control signal to the pinch valve based on the state of the vacuum source, the pinch valve control signal being configured to manipulate a position of the pinch valve.

28. A computer-implemented method for controlling an aspiration system, wherein the aspiration system comprises an aspiration catheter, a vacuum source, a receptacle, an aspiration tubing fluidically coupled to the receptacle and the aspiration catheter, a vacuum line fluidically coupled the receptacle and the vacuum source, a sensor configured to measure a flow parameter associated with a liquid within the aspiration tubing, and a vacuum regulator fluidically coupled to the vacuum line, the computer-implemented method comprising:
receiving the flow parameter from the sensor;
determining a flow condition based on the flow parameter;
setting a characteristic of a pulsed control signal based on the flow condition; and
transmitting the pulsed control signal to the vacuum regulator, the pulsed control signal being configured to control the vacuum regulator to adjust a vacuum level within the vacuum line.

29-43. (canceled)

44. An aspiration system comprising:
an aspiration catheter;
a thrombus retrieval device configured to extend through the aspiration catheter;
a vacuum source; and
an aspiration control system comprising:
a receptacle;
an aspiration tubing configured to fluidically couple the receptacle to an aspiration catheter;
a vacuum line configured to fluidically couple the receptacle to a vacuum source;
a sensor configured to measure a flow parameter associated with a liquid within the aspiration tubing;
a vacuum regulator fluidically coupled to the vacuum line; and
a controller operably coupled to the sensor and the vacuum regulator, wherein the controller is configured to:
receive the flow parameter from the sensor,
determine a flow condition based on the flow parameter,
set a characteristic of a pulsed control signal based on the flow condition, and
transmit the pulsed control signal to the vacuum regulator, the pulsed control signal being configured to control the vacuum regulator to adjust a vacuum level within the vacuum line.
